(19) United States
(12) Patent Application Publication
(10) Pub. No.: US 2024/0197765 A1
(43) Pub. Date: Jun. 20, 2024

(54) Antiviral compositions comprising liposome-encapsulated ascorbyl glucoside and natural extracts

(71) Applicant: BIONEXT LABS LTD, Yavne (IL)

(72) Inventor: Michael Shlomo Bernstein, Rishon LeTsiyon (IL)

(21) Appl. No.: 18/556,171

(22) PCT Filed: May 13, 2021
(86) PCT No.: PCT/IL2021/050553

§ 371 Date: Oct. 19, 2023

(30) Foreign Application Priority Data
May 18, 2020 (IL) 274753

(51) Int. Cl. A61K 31/7056; A61K 9/127; A61K 35/644; A61K 36/185; A61K 36/282; A61K 36/53; A61K 36/537; A61K 36/61; A61K 36/63; A61K 36/8962; A61K 36/9066; A61K 47/10; A61K 47/24

(52) U.S. Cl. CPC A61K 31/7056; A61K 9/127; A61K 35/644; A61K 36/185; A61K 36/282; A61K 36/53; A61K 36/537; A61K 36/61; A61K 36/63; A61K 36/8962; A61K 36/9066; A61K 47/10; A61K 47/24

(57) ABSTRACT

Antiviral liposomal compositions comprising ascorbyl 2-glucoside (AA2G) and other active ingredients including propolis extract and other natural plant extracts, use thereof as an effective medicament, prophylactic, or nutritional supplement against viral infections in general and for treating and or preventing coronavirus disease 2019 (COVID-19) in particular, and methods of preparing such antiviral compositions.

### FIELD OF THE INVENTION

The present invention relates to novel antiviral compositions comprising a vitamin C derivative. More specifically, the invention relates to antiviral liposomal compositions comprising ascorbyl 2-glucoside (AA2G) as a main active ingredient, as well as other active ingredients including propolis extract and other natural plant extracts, and to their use as an effective medicament, prophylactic, or nutritional supplement against viral infections in general and particularly for treating and or preventing coronavirus disease 2019 (COVID-19). The invention further relates to methods of preparing such antiviral compositions.

### BACKGROUND OF THE INVENTION

Viral infections, which occur when pathogenic viruses invade and replicate in the cells of an organism, cause countless life-threatening or life-impairing diseases in both humans and other organisms. In fact, viral diseases are one of the leading causes of human death worldwide, particularly in low-income countries, with viral respiratory diseases being the most frequent cause of death mainly due to infections by influenza viruses. In particular, coronavirus disease 2019 (COVID-19), which is caused by severe acute respiratory syndrome coronavirus 2 (SARS-COV-2, and colloquially referred to as coronavirus) is a contemporary example of an infectious disease that has spread globally leading to an ongoing pandemic, resulting in numerous deaths, a great burden on healthcare systems and significant financial loss across world economies. Despite major progress in the research and treatment of infectious diseases, many viral diseases currently afflicting humans such as COVID-19, still lack a specific treatment (i.e., viral medication), cure or vaccine leaving healthcare providers to manage the disease only by means of supportive care and preventative measures.

It is well recognized in the art that vitamin C (also knowns as ascorbic acid), as well as a number of other vitamins and micronutrients, sometimes in amounts that far exceed the recommended daily levels, can safely and effectively boost the immune system and aid in fighting off viral infections such as COVID-19 and other viral acute respiratory tract diseases. Moreover, as it has been shown to exhibit, inter alia, strong and selective antiviral activity against a number of viruses, vitamin C has frequently been dubbed as a low-cost approach to complementing good hygiene practices and vaccinations efforts in treating and preventing the spread of viral infections. However, in spite of its well-documented antiviral activity in vitro, vitamin C in practice often offers only temporary and limited benefits, if any, to infected individuals. Some of the main reasons for this discrepancy in efficacy stems from the fact that vitamin C has a limited bioavailability due to its poor biochemical and physiological properties. Specifically, vitamin C is preferentially soluble in polar solvents and mainly in water, suffers from significant thermal and oxidative instability in solution leading to appreciable decomposition under physiological settings, has non-optimal absorption rates by cellular transporters, such as intestinal sodium-dependent vitamin C transporters (SVCTs) which absorb the vitamin modestly (about 25%) but only at lower oral doses (far below 200 mg), and, thus, has a poor ability to penetrate and target infected cells so that it rarely comes into contact with the pathogen.

Entrapment or encapsulation of active ingredients into liposomes, which are lipid bilayer micro-vesicles, is a recent approach at attempting to improve the delivery of active agents to infected cells and or reduce toxic effects associated with their administration. As such, with the aim of increasing the bioavailability of ascorbic acid, liposome-encapsulated preparations were reported to substantially increase vitamin C levels in the blood as compared to standard non-encapsulated (non-liposomal) formulations at the same administered dose. However, despite the higher absorption rate afforded by liposomal dosage forms, these strategies still do not address ascorbic acid's inherent instability and insolubility issues, and, hence, fail to concentrate this antiviral agent in its active form within infected cells, which limits their usefulness. In particular, given its hydrophilic character and despite increased intracellular levels achieved by current liposomal formulations, ascorbic acid activity remains restricted to aqueous phases, making it impermeable to lipophilic environments such as the cell membrane, and, hence preventing it from exerting its activity on pathogens residing therein.

Therefore, given the unsuccessful strategies aimed at reaching therapeutic levels of ascorbic acid in infected cells, there continues to be a need for compositions and methods that prove to be efficacious in the delivery, bioavailability, and intracellular targeting of physiologically stable antiviral forms of vitamin C for the prevention and treatment of viral infections in general, and COVID-19 infections in particular. Furthermore, we believe that the sought antiviral activity of bioavailable and physiologically stable vitamin C formulations ought to benefit greatly from the addition of natural extracts having known broad-spectrum antiviral activity, and may lead in many viral infections to synergy between ascorbic acid and other natural extract antiviral activities, further improving the efficacy of treating and or preventing viral diseases, including, but not limited to, COVID-19.

It is therefore an object of the present invention to provide compositions comprising liposome-encapsulated, lipid-soluble vitamin C and other natural extracts, having excellent broad-spectrum antiviral activity, including against SARS-COV-2.

It is another object of the invention to provide said broad-spectrum antiviral composition comprising liposome-encapsulated, lipid-soluble vitamin C and other natural extracts for use as a medicament, prophylactic, or nutritional supplement against viral infections in humans and in other organisms.

It is yet another object of the invention to provide a method of preparing said broad-spectrum antiviral composition comprising liposome-encapsulated, lipid-soluble vitamin C and other natural extracts.

Other objects and advantages of the invention will become apparent as the description proceeds.

### SUMMARY OF THE INVENTION

According to one aspect, the invention provides an antiviral liposomal composition comprising ascorbyl 2-glucoside (AA2G).

According to a specific embodiment, the percentage of AA2G in said composition is between 2-60% (w/w).

According to another specific embodiment, the percentage of AA2G in said composition 15% (w/w).

According to a further embodiment, said composition further comprises propolis extract.

According to a specific embodiment, the percentage of propolis extract in said composition is between 0.15-10.0% (w/w).

According to another specific embodiment, the percentage of propolis extract in said composition is 0.8% (w/w).

According to a further embodiment, said composition further comprises one or more plant extracts or essential oils selected from Artemisia herba-alba, Olea europaea leaf, Thymus vulgaris, Zingiber officinal, Curcuma longa root, Moringa oleifera, Eucalyptus globulus leaf, Allium sativum, Origanum vulgare, Salvia officinalis, Lavandula angustifolia, Rosmarinus officinalis, Eugenia caryophyllata, Leptospermum scoparium, and Carica papaya.

According to a specific embodiment, the percentage of any one or more of said one or more plant extracts or essential oils in said composition is between 0.1-25.0% (w/w).

According to another specific embodiment, the percentage of any one or more of said one or more plant extracts or essential oils corresponds to one or more of the respective percentages from the following table:

| Reagent | % (w/w) |
|---|---|
| White wormwood (Artemisia herba- | 2.2 |
| alba) | |
| Olive (Olea europaea) leaf | 0.3 |
| Blue gum (Eucalyptus globulus) leaf | 0.2 |
| Ginger (Zingiber officinale) | 0.15 |
| Thyme (Thymus vulgaris) | 0.2 |
| Sage (Salvia officinalis) | 2.3 |
| Drumstick tree (Moringa oleifera) | 0.15 |
| Garlic (Allium sativum) | 0.15 |
| Turmeric (Curcuma longa) | 0.15 |
| Lavender (Lavandula angustifolia) | 0.4 |
| Papaya (Carica papaya) | 0.3 |
| Wild oregano (Origanum vulgare) | 0.2 |
| Rosemary (Rosmarinus officinalis) | 0.1 |
| Clove (Eugenia caryophyllata) | 0.2 |

According to another embodiment, the liposome component of said composition is made from ethosomes.

According to a specific embodiment, said ethosomes comprise water, ethanol, propylene glycol and sunflower lecithin.

According to a further specific embodiment, the percentages of water, ethanol, propylene glycol and sunflower lecithin in said ethosomes are between 1-25%.

According to another specific embodiment, the percentages of water, ethanol, propylene glycole and sunflower lecithin in said ethosomes are such that water is 17.8%, ethanol is 12.9%, propylene glycol is 5.2%, and sunflower lecithin 20%.

According to another embodiment, said composition further comprises a pharmaceutically acceptable excipient.

According to a further embodiment, said composition is used in treating or preventing acute or chronic viral infections in a subject, wherein said subject is administered a therapeutically effective dose of the composition.

According to specific embodiment, said acute or chronic viral infection is a viral respiratory infection.

According to another specific embodiment, the said acute or chronic viral infections cause a condition, disorder or disease selected from:
gastroenteritis; keratoconjunctivitis; pharyngitis; croup; pharyngoconjunctival fever; pneumonia; cystitis; hand, foot and mouth disease; pleurodynia; aseptic meningitis; pericarditis; myocarditis; infectious mononucleosis; cytomegalic inclusion disease; Premature birth; liver, lung and spleen diseases newborns; small size at birth; small head size; congenital seizures in the newborn; Burkitt's lymphoma; Hodgkin's lymphoma; nasopharyngeal carcinoma; chronic hepatitis; hepatic cirrhosis; hepatocellular carcinoma; acute hepatitis; hepatocellular carcinoma; herpes labialis, cold sores; gingivostomatitis in children; tonsillitis & pharyngitis in adults; keratoconjunctivitis; AIDS; Kaposi sarcoma; multicentric Castleman disease; primary effusion lymphoma; hyperplastic epithelial lesions (common, flat, plantar and anogenital warts, laryngeal papillomas, epidermodysplasia verruciformis); cervical carcinoma, squamous cell carcinomas; Reye syndrome; postinfectious encephalomyelitis; mumps; croup; pneumonia; bronchiolitis; common cold; poliomyelitis; rabies (fatal encephalitis); post-exposure prophylaxis; influenza-like syndrome; severe bronchiolitis with congenital rubella; German measles; COVID-19; chickenpox; herpes zoster; and congenital varicella syndrome.

According to a further specific embodiment, the said acute viral infection specifically causes COVID-19.

According to another specific embodiment, said subject is a human or another organism.

According to a further specific embodiment, said composition is administered once, or on a regular basis continuously or intermittently.

According to another specific embodiment, said therapeutically effective dose is in the form of a tablet, pill, capsule, cachet, lozenge, granule, semisolid, powder, sustained release formulation, solution, suspension, emulsion, elixir, aerosol or in any other appropriate composition which may or may not include another pharmaceutically acceptable excipient.

According to another embodiment, the invention provides a method of preparing said antiviral liposomal composition, wherein the method comprises blending together said active ingredients and liposomal components into a homogeneous dispersion, entrapping said dispersion into liposomes via mechanical dispersion, and purifying said liposomes from other non-encapsulated material.

### DETAILED DESCRIPTION OF THE INVENTION

The liposomal antiviral compositions according to the invention address the need for preventing, controlling, treating and eradicating viral diseases that afflict humans and other organisms, such as but not limited to COVID-19. The invention is based on the realization that biologically-stabilized, lipid-soluble forms of vitamin C, and namely ascorbyl 2-glucoside (AA2G), are useful, in conjunction with other naturally-derived antiviral extracts, as broad-spectrum antiviral agents when administered in a particular liposomal formulation.

The term “broad-spectrum antiviral agent” is used herein to refer to any antimicrobial substance or composition thereof which has well-known therapeutic properties in relation to viral infections, and, hence, can be used as a medicament, prophylactic, or supplement to treat a wide range of conditions, disorders or diseases caused by various chronic or acute viral infections in mammals, such as humans, and in plants. Such conditions, disorders and diseases include, but are not limited to gastroenteritis, keratoconjunctivitis, pharyngitis, croup, pharyngoconjunctival fever, pneumonia, cystitis, hand, foot and mouth disease, pleurodynia, aseptic meningitis, pericarditis, myocarditis, infectious mononucleosis, cytomegalic inclusion disease, premature birth, liver, lung and spleen diseases newborns, small size at birth, small head size, congenital seizures in the newborn, Burkitt's lymphoma, Hodgkin's lymphoma, nasopharyngeal carcinoma, chronic hepatitis, hepatic cirrhosis, hepatocellular carcinoma, acute hepatitis, hepatocellular carcinoma, herpes labialis, cold sores, gingivostomatitis in children, tonsillitis & pharyngitis in adults, keratoconjunctivitis, AIDS, Kaposi sarcoma, multicentric Castleman disease, primary effusion lymphoma, hyperplastic epithelial lesions (common, flat, plantar and anogenital warts, laryngeal papillomas, epidermodysplasia verruciformis), cervical carcinoma, squamous cell carcinomas, Reye syndrome, postinfectious encephalomyelitis, mumps, croup, pneumonia, bronchiolitis, common cold, poliomyelitis, rabies (fatal encephalitis), Post-exposure prophylaxis, influenza-like syndrome, severe bronchiolitis with congenital rubella, German measles, COVID-19, chickenpox, herpes zoster, and congenital varicella syndrome. AA2G, also known by various alternate names in the art including ascorbyl glucoside, ascorbyl 2-glucoside, 2-O-α-ascorbyl glucoside, L-ascorbic acid 2-O-glucoside, and 2-O-alpha-D-glucopyranosyl-L-ascorbic acid, (CAS No. 129499-78-1), is different than conventional vitamin C in that it is an amphiphilic, and hence also detergent-soluble, pro-vitamin glucoside derivative of ascorbic acid having longer lasting effects compared to the physiological vitamer owing to its increased solubility and stability in various environments. Importantly, the inventors of the instant application have unexpectedly found that AA2G, if administered properly, could potentially lead to greater effective concentrations of vitamin C in the blood as well as throughout cells, and, hence, boost the immune system to thwart viral infections or effectively fight them off. Strikingly, there are no reports of using this pro-vitamin C derivative, exclusively or in combination with other natural antiviral substances, directly against viral infections.

As other biologically relevant glucoside forms of vitamin C are known in the art, such as the B-anomeric 2-O-β-D-glucopyranosyl-L-ascorbic acid (AA-2β-G) isolated from Goji berries (Lycium barbarum L.), for the sake of clarity and to avoid confusion with these other forms, AA2G as defined herein refers to the alpha glycosidic form, as well as to (pharmaceutically acceptable) salts thereof, having the following parent chemical structure:

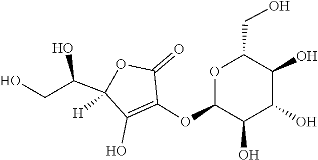 [SMILES: O=C1O[C@H]([C@H](O)CO)C(O)=C1O[C@H]1O[C@H](CO)[C@@H](O)[C@H](O)[C@H]1O]

Nevertheless, a skilled artisan would appreciate that under particular circumstances AA2G may be substituted with other glucoside forms (e.g., AA-2β-G)), and any lipophilic ascorbyl derivatives for that matter, without losing appreciable efficacy and possibly gaining additional properties specific to the desired outcome (e.g., formulating lipid-specific compositions which preferentially target membranes).

The terms “antiviral” or “antiviral agents” as applied herein may further encompass, in addition to substances that possess antiviral properties either in vivo or in vitro, anti-infective agents, antibiotics, antiseptics, antimicrobials, and any natural therapeutic substance that exerts anti-pathological effects.

The terms “nutraceutical”, “nutritional supplement” or “dietary supplement” are used interchangeably herein to refer to any product that is intended to provide essential nutrients, especially in the context of boosting immune function, which may otherwise not be consumed or absorbed in sufficient quantities, such as vitamin C. Said products are typically ingested in capsule, tablet or liquid form, and may further include but are not limited to species which are antioxidants, antiradicals or radical scavengers, and scavengers of reactive oxygen species.

In addition to increasing blood and intracellular therapeutic levels of vitamin C in cells as described further herein below, the unique liposome-based formulations devised by the inventors of the instant application can theoretically carry and effectively deliver ascorbic acid, as well as other natural antiviral agents as disclosed herein, directly into certain viruses, such as via membrane fusion of the liposomal carrier with the outer phospholipid bilayer, or the envelope, of most viruses. As such, the compositions of the invention may concentrate the sugar-vitamin conjugate directly in or around the virally infected site further improving the efficacy of the treatment.

A skilled artisan will surely appreciate that although a specific type of liposome is employed in the formulations herein (i.e., ethosomes, which are typically composed of ethanol, glycols, water, and phospholipids such as those found in lecithin), many other liposomal variants can be envisioned and utilized to afford effective antiviral AA2G-containing liposomal compositions. Furthermore, it should be realized that other liposomal delivery systems could offer additional benefits, for instance, allowing additional pharmaceutically acceptable excipients to be included, better protection against enzymatic degradation, improved pharmacokinetics and tissue distribution, and a controlled release of therapeutic agents to appropriate cells. In addition, it should be well known to the skilled person that the distribution and therapeutic availability of liposomes can be modulated through variations of their size, lamellarity, lipid composition, charge and surface properties. It is therefore a fundamental concept in the art to adapt the physicochemical properties of liposomes with the desired therapeutic objective. Furthermore, because of the similarity of the primary components of liposomes with natural membranes, liposomes are generally non-toxic and biodegradable, and thus, liposomal preparations as disclosed by the instant invention exhibit these same useful benefits.

It is also worth noting that the skilled artisan will further appreciate that although the liposomal formulations disclosed herein were prepared by ultrasonication, any number of mechanical dispersion methods that are known in the art would suffice for preparing the liposomal compositions of the invention or any similar antiviral liposomal composition for that matter, which include but are not limited to French pressure cell (extrusion), freeze-thawed liposomes, lipid film hydration by hand shaking, non-hand, shaking or freeze drying, micro-emulsification, membrane extrusion, and dried reconstituted vesicles.

Further to the provision of antiviral AA2G-loaded liposomal compositions as described above, the inventors have also identified additional natural sources of well-documented antiviral agents, which, when combined in liposomal formulations as disclosed herein, ought to complement and synergize with ascorbic acid's antiviral activity. These natural antiviral auxiliaries may include extracts and essential oils from animal-made products such as from propolis, as well as from various plants including white wormwood and olive leaf. It is worth noting that AA2G preparation containing these natural substances have never been disclosed together in a single preparation, and especially never in the context of antiviral or virus-targeted compositions.

The term “propolis”, colloquially known as bee glue, as used herein refers to a resinous mixture that honey bees produce by mixing saliva and beeswax with exudate gathered from tree buds, sap flows, or other botanical sources.

The term “Artemisia herba-alba”, known colloquially as white wormwood or shiba, as used herein may also refer to other species from the genus Artemisia, including, for example, Artemisia annua (sweet wormwood).

The terms “extract”, and “oil” or “essential oil” are used in the instant application to refer to an aqueous-organic dispersion and concentrated hydrophobic liquid, respectively, containing organic compounds from plants or other natural sources. Essential oils are also known as volatile oils, ethereal oils, and as the oil of the plant from which they were extracted, such as but not limited to oils of wormwood, Manuka (Leptospermum scoparium), oregano, and thyme.

According to specific embodiments, the instant invention provides antiviral liposomal compositions comprising the antiviral agents disclosed herein in various combinations percentages which, as the skilled person will surely realize, are not solely confined to those demonstrated in the examples herein, but, rather, encompass a larger set of reasonable values as may be contemplated by those of skill in the art. Specifically speaking, it should be evident to a skilled artisan from the exemplary formulations described further herein that the reagent combinations and percentages used during the formulation process may be varied altogether or each one independently, viz., in a mutually inclusive or exclusive manner to produce a series of antiviral liposomal compositions without departing from the spirit and intended scope of the invention.

Accordingly, antiviral compositions according to the invention may comprise, in addition to the combinations and percentages of reagents presented in the examples described further below, only a representative sample of all the possible combination concentration of reagents employed in preparing the compositions of the invention, and , hence, a range of reagent percentages may be envisioned. 5-25% AA25 with a preference for 15% AA2G, propolis may comprise.

The terms “percentage” (i.e., %), “concentration”, “fraction”, “level” or “abundance” are used interchangeably herein to refer to the final amount (in units of grams) of a given substance divided by the total amount of all substances (including solvents) in a given composition. This interpretation is essentially identical to the definition of the mass fraction of a given constituent in a composition, which is conveniently represented herein as % (w/w).

It is a main object of the present invention to provide liposomal formulations of ascorbyl glycoside, with and without additional natural supplements, which deliver vitamin C directly to needed areas in the body, and hence could be used to prevent and treat various viral diseases, such as, but not limited to, COVID-19. Such targeted delivery system result in an increased efficacy and reduced healing time in both humans and other organisms, such as livestock, and rare zoo animals. In addition, the improved bioavailability of these antivirals upon encapsulation into liposomes could reduce the dosing interval and amount administered, consequently improving the quality of life of individuals infected with debilitating viral infections.

Although the description refers mainly to therapeutic applications of the disclosed formulations, for the sake of simplicity, it should be noted that the instant invention is not limited to medicaments, and, rather, it can be utilized as a prophylactic agent, nutritional supplement, and even a disinfectant in certain cases (e.g., sprayed on inanimate objects and plants to control the spread of viruses or other microbial pathogens). Accordingly, another important aspect of the invention concerns the provision of natural liposome-based nutritional supplements and disinfectants to aid in boosting the immune system and fighting off a broad range of viral infections, such as those which cause COVID-19.

In general, compositions of this invention (antiviral AA2G-containing liposomal compositions) may be administered akin to ordinary pharmaceutical or nutraceutical compositions by one of the following routes: oral, topical, systemic (e.g. transdermal, intranasal, intrapulmonary or by suppository), parenteral (e.g. intramuscular, subcutaneous, intra-arterial, intraperitoneal or intravenous injection), by implantation and by infusion through such devices as osmotic pumps, transdermal patches and the like. Compositions may also take the form of tablets, pills, capsules, cachets, lozenges, granules, semisolids, powders, sustained release formulation, solutions, suspensions, emulsions, elixirs, aerosols or any other appropriate compositions which may or may not include additional pharmaceutically acceptable excipients.

The terms “physiological”, “physiologically” or “physiologically-relevant” are used interchangeably herein to mean that the element in question (e.g., biomolecule, liposome, composition, etc.) has biochemical or functional characteristics that correspond to or mimic those of healthy or normal biological systems.

Reference will now be made to several detailed embodiments of the present invention, such as the examples detailed further herein below. It should be noted that the following examples depict embodiments of the present invention for illustration purposes only. That is, the examples are representative of the materials and techniques employed by the inventors in carrying out particular aspects of the present invention. Also, it should be appreciated that while these materials and techniques are exemplary of specific embodiments of how to practice the invention, those of skill in the art, in light of the present disclosure, will recognize that numerous modifications can be envisioned without departing from the spirit and intended scope of the invention.

### EXAMPLES

### Materials and Methods

All the starting materials used in the preparation of formulations disclosed herein are naturally and locally sourced, except where specifically stated to the contrary. Namely, all dried plants and propolis were purchased from local suppliers (e.g., Al-Alim, Israel) which grow or produce the product under control conditions without pesticides, and were extracted with conventional, natural or plant-derived solvent systems of water/glycerin, water/propylene glycol or water/alcohol (supplied by Bio-Lab Ltd., Israel). Solid ascorbyl 2-glucoside was purchased from Spec-Chem Industry Inc., China (purity ≥98.0%). All plant essential oils, fats and extracts derived from plants and apiculture were either purchased in high quality/purity grade from Israeli suppliers or importers, or obtained with the use of the above-mentioned solvents via extraction methods that are well-known in the art, such as but not limited to steam distillation, expression, solvent extraction, sfumatura, absolute oil extraction, resin tapping, wax embedding, and cold pressing. In addition, all other solvents used in the preparations of the disclosed formulations are either naturally sourced, plant-derived or plant-based, and include but are not limited to demineralized water, glycerin, propylene glycol, and ethanol.

### Example 1

### Preparation of Antiviral Formulations

A specific objective of the instant invention is to provide effective broad-spectrum antiviral compositions comprising the detergent-soluble, physiologically stable form of vitamin C, ascorbyl 2-glucoside (AA2G), as well as ones combined with concentrated natural extracts from propolis and other plants with proven antiviral, antimicrobial, anti-inflammatory and other medicinal properties, such as but not limited to Carica papaya extract which is known to have proteolytic enzymatic activity toward viral capsid constituents, and have heretofore never been disclosed together in such a unique bioavailable liposomal formulation.

In particular, the inventors of the instant application have serendipitously found that instead of using the standard water/alcohol solvent system for the liposomal synthesis step and subsequently encapsulating desired active ingredients therein, a solution comprising, in addition to the standard solvents above, the unique blend of antiviral substances to be incorporated can be used directly during the liposomal formation step to afford higher and more physiological-relevant loadings of active ingredients, presumably entrapped throughout the fat and aqueous phases of the vesicle. Hence, this novel preparation method yields more potent, fast acting, and versatile antiviral formulations.

Thus, on the basis of the foregoing, representative antiviral compositions according to the instant invention were prepared as follows:

First, as noted above, each of the plant extracts, which were used as reagents during the formulation step, were prepared via solvent extraction by (1) suspending the dried plant forms (e.g., dried herbs) in a solvent system comprising at least one solvent selected from water, glycerin, propylene glycol and ethanol, as indicated in the formulation tables below, (2) extracting the oil by heating the suspension at 50° C. for 1 hour and letting the mixture sit at room temperature for 10 days, and (3) removing solid plant material via any number of filtration and centrifugation techniques well-known to the skilled person. If the final formulation comprised at least one other antiviral reagent in addition to AA2G, then AA2G and said at least one antiviral reagent selected from extracts of propolis or plant, or plant essential oils, as indicated in the formulation tables below, were blended in a commercial-grade homogenizer (Silverson) for 30 minutes to yield a dispersion. Next, to said dispersion, or to an aqueous solution of AA2G if the final formulation comprised AA2G as the sole antiviral reagent, sunflower lecithin was added, and optionally water, ethanol or propylene glycol were added as necessary to attain the final concentrations as determined from the formulation tables below, and the solution was transferred to a ultrasonifier for preparation of and entrapment of the dispersion in liposomes (i.e., ethosomes) as conventionally done in the art. The loaded ethosomes, which are uni- or multi-lamellar and range in size anywhere from 30 nm to a few microns, were isolated from the non-encapsulated material via any number of purification techniques such as but not limited to differential centrifugation, dialysis and various analytical chromatographic techniques that are all well established in the art. The resulting liposomal formulations were stable for at least 3 months at 4° C., and could be orally administered in the form of tablets, pills, capsules, cachets, lozenges, granules, semisolids, powders, sustained release formulation, solutions, suspensions, emulsions, elixirs, aerosols or in any other appropriate composition which may or may not include other pharmaceutically acceptable excipients, with the preparation of such administration forms being well-known to the skilled artisan.

TABLE 1
Formulation A

| | | % of | grams |
|---|---|---|---|
| | % of | active | per |
| | reagent | ingredient | kilo |
| Reagent | (w/w) | (w/w) | gram |
| Solids | | | |
| Ascorbyl 2-glucoside | 15 | 15 | 150 |
| Sunflower lecithin | 20 | 20 | 200 |
| Solvents | | | |
| Propylene glycol | 5.2 | | 52 |
| Ethanol | 12.9 | | 129 |
| Water | 46.9 | | 469 |

TABLE 2
Formulation B

| | | % of | grams |
|---|---|---|---|
| | % of | active | per |
| | reagent | ingredient | kilo |
| Reagent | (w/w) | (w/w) | gram |
| Extracts | | | |
| 10% Propolis in 100% propylene glycol | 3 | 0.3 | 30 |
| 10% Propolis in 96% ethanol/4% water | 5 | 0.5 | 50 |
| Solids | | | |
| Ascorbyl 2-glucoside | 15 | 15 | 150 |
| Sunflower lecithin | 20 | 20 | 200 |
| Solvents | | | |
| Propylene glycol | 5.2 | | 52 |
| Ethanol | 12.9 | | 129 |
| Water | 38.9 | | 389 |

TABLE 3
Formulation C

| | | % of | grams |
|---|---|---|---|
| | % of | active | per |
| | reagent | ingredient | kilo |
| Reagent | (w/w) | (w/w) | gram |
| Extracts | | | |
| 10% White wormwood (Artemisia herba-alba) | 21 | 2.1 | 210 |
| in 95% water/5% glycerin or 5% propylene | | | |
| glycol | | | |
| 10% Propolis in 100% propylene glycol | 3 | 0.3 | 30 |
| 10% Propolis in 96% ethanol/4% water | 5 | 0.5 | 50 |
| Essential Oils | | | |
| White wormwood (Artemisia herba-alba) | 0.1 | 0.1 | 1 |
| Solids | | | |
| Ascorbyl 2-glucoside | 15 | 15 | 150 |
| Sunflower lecithin | 20 | 20 | 200 |
| Solvents | | | |
| Propylene glycol | 5.2 | | 52 |
| Ethanol | 12.9 | | 129 |
| Water | 17.8 | | 178 |

TABLE 4
Formulation D

| | | % of | grams |
|---|---|---|---|
| | % of | active | per |
| | reagent | ingredient | kilo |
| Reagent | (w/w) | (w/w) | gram |
| Extracts | | | |
| 10% White wormwood (Artemisia herba- | 21 | 2.1 | 210 |
| alba) in 95% water/5% glycerin or | | | |
| 5% propylene glycol | | | |
| 10% Sage (Salvia officinalis) in 95% | 23 | 2.3 | 230 |
| water/5% propylene glycol | | | |
| 10% Propolis in 100% propylene glycol | 3 | 0.3 | 30 |
| 10% Propolis in 96% ethanol/4% water | 5 | 0.5 | 50 |
| 10% Olive (Olea europaea) leaf in 67.2% | 3 | 0.3 | 30 |
| ethanol/32.8% water | | | |
| 5% Drumstick tree (Moringa oleifera) in | 3 | 0.15 | 30 |
| 67.2% ethanol/32.8% water | | | |
| 5% Garlic (Allium sativum) in 67.2% | 3 | 0.15 | 30 |
| ethanol/32.8% water | | | |
| 5% Turmeric (Curcuma longa) in 67.2% | 3 | 0.15 | 30 |
| ethanol/32.8% water | | | |
| Essential Oils | | | |
| Lavender (Lavandula angustifolia) | 0.4 | 0.4 | 4 |
| White wormwood (Artemisia herba-alba) | 0.1 | 0.1 | 1 |
| Wild oregano (Origanum vulgare) | 0.2 | 0.2 | 2 |
| Rosemary (Rosmarinus officinalis) | 0.1 | 0.1 | 1 |
| Clove (Eugenia caryophyllata) | 0.2 | 0.2 | 2 |
| Solids | | | |
| Ascorbyl 2-glucoside | 15 | 15 | 150 |
| Sunflower lecithin | 20 | 20 | 200 |

TABLE 5
Formulation E

| | % of | % of active | grams |
|---|---|---|---|
| | reagent | ingredient | per kilo |
| Reagent | (w/w) | (w/w) | gram |
| Extracts | | | |
| 10% Propolis in 100% propylene glycol | 3 | 0.3 | 30 |
| 10% Propolis in 96% ethanol/4% water | 5 | 0.5 | 50 |
| 10% Olive (Olea europaea) leaf in 90% | 3 | 0.3 | 30 |
| water/10% glycerin | | | |
| 10% White wormwood (Artemisia herba- | 22 | 2.2 | 220 |
| alba) in 90% water/10% propylene glycol | | | |
| 5% Blue gum (Eucalyptus globulus) leaf in | 4 | 0.2 | 40 |
| 90% water/5% glycerin | | | |
| 5% Drumstick tree (Moringa olifera) in | 3 | 0.15 | 30 |
| 90% water/10% glycerin | | | |
| 5% Ginger (Zingiber officinale) in 90% | 3 | 0.15 | 30 |
| water/10% propylene glycol | | | |
| 5% Turmeric (Curcuma longa) in 90% | 3 | 0.15 | 30 |
| water/9.5% ethanol | | | |
| 5% Thyme (Thymus vulgaris) in 30% | 4 | 0.2 | 40 |
| water/66.5% ethanol | | | |
| 5% Garlic (Allium sativumin) 30% | 3 | 0.15 | 30 |
| water/66.5% ethanol | | | |
| 10 % Papaya (Carica papaya) | 3 | 0.3 | 30 |
| in 90% water/10% propylene glycol | | | |
| Solids | | | |
| Ascorbyl 2-glucoside | 15 | 15 | 150 |
| Sunflower lecithin | 20 | 20 | 200 |
| Solvents | | | |
| Propylene glycol | 1 | | 10 |
| Ethanol | 8 | | 80 |

1. An antiviral liposomal composition comprising ascorbyl 2-glucoside (AA2G), wherein the liposome component of the composition is made from ethosomes.

2. The antiviral liposomal composition of claim 1, wherein the percentage of AA2G in the composition is between 2-60% (w/w).

3. The antiviral liposomal composition of claim 1, wherein the percentage of AA2G in the composition is 15% (w/w).

4. The antiviral liposomal composition of claim 1, wherein the composition further comprises propolis extract.

5. The antiviral liposomal composition of claim 4, wherein the percentage of propolis extract in the composition is between 0.15-10.0% (w/w).

6. The antiviral liposomal composition of claim 4, wherein the percentage of propolis extract in the composition is 0.8% (w/w).

7. The antiviral liposomal composition of claim 1, wherein the composition further comprises one or more plant extracts or essential oils selected from Artemisia herba-alba, Olea europaea leaf, Thymus vulgaris, Zingiber officinal, Curcuma longa root, Moringa oleifera, Eucalyptus globulus leaf, Allium sativum, Origanum vulgare, Salvia officinalis, Lavandula angustifolia, Rosmarinus officinalis, Eugenia caryophyllata, Leptospermum scoparium, and Carica papaya.

8. The antiviral liposomal composition of claim 7, wherein the percentage of any one or more of said one or more plant extracts or essential oils is between 0.1-25.0% (w/w).

9. The antiviral liposomal composition of claim 7, wherein the percentage of any one or more of said one or more plant extracts or essential oils corresponds to one or more of the respective percentages from the following table:

| Reagent | % (w/w) |
|---|---|
| White wormwood (Artemisia herba-alba) | 2.2 |
| Olive (Olea europaca) leaf | 0.2 |
| Blue gum (Eucalyptus globulus) leaf | 0.2 |
| Ginger (Zingiber officinale) | 0.15 |
| Thyme (Thymus vulgaris) | 0.2 |
| Sage (Salvia officinalis) | 2.3 |
| Drumstick tree (Moringa oleifera) | 0.15 |
| Garlic (Allium sativum) | 0.15 |
| Turmeric (Curcuma longa) | 0.15 |
| Lavender (Lavandula angustifolia) | 0.4 |
| Papaya (Carica papaya) | 0.3 |
| Wild oregano (Origanum vulgare) | 0.2 |
| Rosemary (Rosmarinus officinalis) | 0.1 |
| Clove (Eugenia caryophyllata) | 0.2 |

10. The antiviral liposomal composition of claim 1. wherein the ethosomes comprise water, ethanol, propylene glycol and sunflower lecithin.

11. The antiviral liposomal composition of claim 10, wherein the percentages of water, ethanol, propylene glycol and sunflower lecithin are between 1-25%.

12. The antiviral liposomal composition of claim 10, wherein the percentages are such that water is 17.8%, ethanol is 12.9%, propylene glycol is 5.2%, and sunflower lecithin 20%.

13. The antiviral liposomal composition of claim 1, wherein the composition further comprises a pharmaceutically acceptable excipient.

14. The antiviral liposomal composition of claim 1, for use in treating or preventing acute or chronic viral infections in a subject, wherein said subject is administered a therapeutically effective dose of the composition.

15. The antiviral liposomal composition according to claim 14, wherein the acute or chronic viral infection is a viral respiratory infection.

16. The antiviral liposomal composition according to claim 14, wherein the acute or chronic viral infections cause a condition, disorder or disease selected from: gastroenteritis; keratoconjunctivitis; pharyngitis; croup; pharyngoconjunctival fever; pneumonia; cystitis; hand, foot and mouth disease; pleurodynia; aseptic meningitis; pericarditis; myocarditis; infectious mononucleosis; cytomegalie inclusion disease; Premature birth; liver, lung and spleen diseases newborns; small size at birth; small head size; congenital seizures in the newborn; Burkitt's lymphoma; Hodgkin's lymphoma; nasopharyngeal carcinoma; chronic hepatitis; hepatic cirrhosis; hepatocellular carcinoma; acute hepatitis; herpes labialis, cold sores; gingivostomatitis in children; tonsillitis & pharyngitis in adults; AIDS; Kaposi sarcoma; multicentric Castleman disease; primary effusion lymphoma; hyperplastic epithelial lesions (common, flat, plantar and anogenital warts, laryngeal papillomas, epidermodysplasia verruciformis); cervical carcinoma, squamous cell carcinomas; Reye syndrome; postinfectious encephalomyelitis; mumps; bronchiolitis; common cold; poliomyelitis; rabies (fatal encephalitis); post-exposure prophylaxis; influenza-like syndrome; severe bronchiolitis with congenital rubella; German measles; COVID-19; chickenpox; herpes zoster; and congenital varicella syndrome.

17. The antiviral liposomal composition according to claim 14, wherein the acute viral infection specifically causes COVID-19.

18. The antiviral liposomal composition according to claim 14, wherein the subject is a human or another organism.

19. The antiviral liposomal composition according to claim 14, wherein the composition is administered once, or on a regular basis continuously or intermittently.

20. The antiviral liposomal composition according to claim 14, wherein said therapeutically effective dose is in the form of a tablet, pill, capsule, cachet, lozenge, granule, semisolid, powder, sustained release formulation, solution, suspension, emulsion, elixir, aerosol or in any other appropriate composition which may or may not include another pharmaceutically acceptable excipient.

21. A method of preparing an antiviral liposomal composition according to claim 1, the method comprising blending together said active ingredients and liposomal components into a homogeneous dispersion, entrapping said dispersion into liposomes via mechanical dispersion, and purifying said liposomes from other non-encapsulated material.
